(19) United States
(12) Patent Application Publication
(10) Pub. No.: US 2022/0218118 A1
(43) Pub. Date: Jul. 14, 2022

(54) MATTRESS CARE DEVICE

(71) Applicant: LG ELECTRONICS INC., Seoul (KR)

(72) Inventors: JiHyun YOON, Seoul (KR); Hyun Shin KEE, Seoul (KR); Ki Sang JUNG, Seoul (KR); Taihong KIM, Seoul (KR)

(73) Assignee: LG ELECTRONICS INC., Seoul (KR)

(21) Appl. No.: 17/566,328

(22) Filed: Dec. 30, 2021

(30) Foreign Application Priority Data
Jan. 14, 2021 (KR) 10-2021-0005139

(51) Int. Cl. A47C 31/00; A47L 7/00; A47L 9/22; A47L 9/14; A61L 2/10

(52) U.S. Cl. CPC A47C 31/00; A47L 7/008; A61L 2202/26; A47L 9/1409; A61L 2/10; A47L 9/22

(57) ABSTRACT

A mattress care device includes two vertically moving frames that are moved upward from opposite side walls of a bed frame and then a sanitary module is moved along the two vertically moving frames to perform the cleaning of a mattress, and when the cleaning of the mattress is not performed, the sanitary module is stored in a storage part while the two vertically moving frames are moved downward so that the sanitary module is stored in a location hidden from a user of a bed. Accordingly, discomfort caused by exposure of the sanitary module can be prevented.

### CROSS REFERENCE TO RELATED APPLICATION

This application claims the priority benefit of Korean Patent Application No. 10-2021-0005139, filed on Jan. 14, 2021 in the Republic of Korea, which is hereby incorporated by reference in its entirety for all purposes as if fully set forth herein.

### BACKGROUND OF THE INVENTION

### 1. Field of the Invention

The present disclosure relates generally to a new type mattress care device configured to be provided to a bed and to automatically perform sterilization of a mattress in the bed.

### 2. Description of the Background Art

In general, a mattress is used as a support for bedding on the floor or on a bed.

The mattress used for the bed is provided in a state of being placed on a bed frame, and a user can sleep by covering an upper surface of the mattress with bedding.

The above-mentioned mattress for a bed is provided in a state of being permanently provided in the bed frame. Therefore, continuous management for cleaning is required.

Conventionally, periodic washing of a mattress cover, dusting off dust accumulated on the mattress, and cleaning with a bedding vacuum are used as a method of cleaning the mattress, which is disclosed in Korean Patent No. 10-0661619, Korean Patent No. 10-0518620, etc.

The conventional cleaner described above is configured to perform sterilization cleaning using a sterilization lamp (e.g., an ultraviolet-C (UV-C) lamp).

However, in sterilization cleaning using the sterilization lamp, although the sterilization effect may be obtained only when the UV-C light source is radiated for a sufficient time, sufficient sterilization of the mattress may not be achieved as the user performed sterilization and cleaning at the same speed as during general vacuum cleaning.

Furthermore, as disclosed in related art, such as Korean Utility Model Registration No. 20-0388045, Korean Patent Application Publication No. 10-2009-0111632, Korean Patent No. 10-1767055, Korean Patent No. 10-1853624, Korean Patent Application Publication No. 10-2020-0067464, etc., sterilization cleaning for a mattress can be performed with a sterilization device, which has an UV-C lamp at a lower surface thereof and is provided at an upper portion of the mattress to be movable in forward and rearward directions (longitudinal direction of bed).

However, the user may feel discomfort while the sterilization device according to the above-described related art is provided in a structure of being exposed to the outside of the bed. Because the cleaner is present in the sleeping place, the user may feel (i.e., determine) that a smell (odor) is emitted in the bed even if the smell does not actually occur, and the feeling of the user causes the discomfort.

Furthermore, as the sterilization device is exposed to the outside of the bed, interference may occur when the user takes on and off the bed.

In addition, in the related art, the sterilization device is configured to be operated in a state of being provided on an outer surface of the bed frame or in a state of being provided by penetrating the bed frame. As an exposed portion or a penetration portion of the sterilization device is exposed outward, design aesthetics of the bed may deteriorate, and design improvement is difficult.

Furthermore, when the mattress is replaced with a different type of mattress, a distance between the conventional sterilization device and a surface of the new mattress is excessively increased or decreased, and the cleaning effect is reduced.

Specifically, considering that the irradiation distance of UV-C is affected by output and irradiation time, when the irradiation distance of UV-C is not constant, the sterilization effect is rapidly reduced.

The conventional sterilization device has the same length as the width of the mattress and is configured to clean while being moved only in the forward and rearward directions of the mattress, so that it is inevitable to use a motor with a large capacity so as to provide sufficient suction power.

Specifically, a lamp radiating UV-C also includes a plurality of lamps, so that overall manufacturing cost is inevitably high.

### DOCUMENTS OF RELATED ART

(Patent Document 0001) Korean Patent No. 10-0661619.
(Patent Document 0002) Korean Patent No. 10-0518620.
(Patent Document 0003) Korean Patent No. 10-1767055.
(Patent Document 0004) Korean Patent No. 10-1853624.
(Patent Document 0005) Korean Patent Application Publication No. 10-2020-0067464.

### SUMMARY OF THE INVENTION

Accordingly, the present disclosure has been made keeping in mind the above problems occurring in the related art, and the present disclosure is intended to provide a new type mattress care device configured to efficiently perform sterilization cleaning for a mattress and to be stored while being hidden from the outside in an unused state thereof.

Another objective of the present disclosure is intended to provide a new type mattress care device, which is configured to perform UV-C irradiation while being placed as close to a mattress as possible, resulting in a stable sterilization effect. However, any type of sterilizing irradiation may be used, such as Far-UV.

Further objective of the present disclosure is intended to provide a new type mattress care device, which is configured to perform efficient cleaning for an entire mattress only by a small suction force or a small UV-C irradiation area.

In order to achieve the above objectives, according to one aspect of the present disclosure, a mattress care device may be configured such that when cleaning for a mattress is performed, two vertically moving frames are moved upward from opposite side walls of a bed frame and then a sanitary module is moved along the vertically moving frames to clean the mattress. Therefore, when the mattress cleaning is not performed, the sanitary module may be stored in a location hidden from the view of a bed user.

According to the present disclosure, the mattress care device may include the bed frame having a mattress accommodation part storing the mattress and a storage part providing a storage space.

According to the mattress care device of the present disclosure, the storage part may include a storage groove formed on an upper surface of at least one of a front end or a rear end of the bed frame.

According to the mattress care device of the present disclosure, the vertically moving frames may be provided to be movable in vertical directions at opposite side walls of the bed frame.

According to the mattress care device of the present disclosure, the two vertically moving frames may have at least length reaching a front end to a rear end of the mattress.

The mattress care device of the present disclosure may include a sanitary module configured to clean the mattress.

According to the mattress care device of the present disclosure, the sanitary module may be configured such that opposite ends thereof are respectively connected to the two vertically moving frames to be movable in forward and rearward directions along the two vertically moving frames.

According to the mattress care device of the present disclosure, the sanitary module may be stored in the storage part of the bed frame when not in use.

According to the mattress care device of the present disclosure, the sanitary module may be configured to be movable in the forward and rearward directions by a moving part.

According to the mattress care device of the present disclosure, the sanitary module may be configured to be operated under control of a controller.

According to the mattress care device of the present disclosure, a through installation part having a vertical through hole may be provided at each corner of the bed frame.

According to the mattress care device of the present disclosure, vertical ends in the vertically moving frames may be formed by being bent downwardly from a front end and a rear end of each of the vertically moving frames to pass through the through hole of the through installation part.

The mattress care device of the present disclosure may include a frame elevation part operated under the control of the controller and configured to raise and lower the vertically moving frames.

According to the mattress care device of the present disclosure, the frame elevation part may be configured to raise the vertically moving frames when cleaning operation is performed, so that the sanitary module may be located higher than the upper surface of the mattress.

According to the mattress care device of the present disclosure, the frame elevation part may be configured to lower the vertically moving frames when the cleaning operation is not performed, so that the sanitary module may be located lower than the upper surface of the mattress.

According to the mattress care device of the present disclosure, the frame elevation part may include a ball screw (i.e., a first ball screw) coupled to an associated vertical end and an elevating motor provided for rotation driving of the ball screw.

According to the mattress care device of the present disclosure, the ball screw may be configured to be operated in conjunction with another ball screw (i.e., a second ball screw) by an interlocking device.

According to the mattress care device of the present disclosure, the storage part may include the storage groove formed by being depressed from an upper surface of at least one of the front end or the rear end of the bed frame.

According to the mattress care device of the present disclosure, the sanitary module may be configured to be inserted and stored in the storage groove.

According to the mattress care device of the present disclosure, the sanitary module may include a movable bar of which opposite ends are respectively connected to the two vertically moving frames and configured to be movable in the forward and rearward directions.

According to the mattress care device of the present disclosure, the sanitary module may include a sterilizing cleaner configured to be movable in leftward and rightward directions along a lower surface of the movable bar.

According to the mattress care device of the present disclosure, the left to right length of the sterilizing cleaner may be configured shorter than the left to right length of the movable bar.

According to the mattress care device of the present disclosure, an inlet may be provided on a bottom surface of the sanitary module, the sanitary module may include a fan motor and a dust collecting casing therein, the fan motor being configured to generate a vacuum suction force to the inlet, and the dust collecting casing being configured to collect debris contained in air suctioned through the inlet.

According to the mattress care device of the present disclosure, an UV-C irradiation part may be provided on the bottom surface of the sanitary module.

According to the mattress care device of the present disclosure, the moving part may include a rack gear provided on each of facing surfaces between the opposite vertically moving frames in a longitudinal direction of an associated vertically moving frame, and a pinion gear provided at each of opposite sides of the sanitary module.

According to the mattress care device of the present disclosure, a guide part may be provided on each of the facing surfaces between the vertically moving frames and be configured to guide movement of the sanitary module.

According to the mattress care device of the present disclosure, the guide part may be configured to protrude in an inward direction from each of the facing surfaces of the opposite vertically moving frames.

According to the mattress care device of the present disclosure, and each of the opposite ends of the sanitary module may be configured to be placed on the guide part.

As described above, the mattress care device of the present disclosure has following various effects.

The mattress care device of the present disclosure has the storage part in the bed frame to provide a storage space, so that the mattress care device cannot be exposed (i.e., is not exposed) to the outside when the sanitary module is not used.

The mattress care device of the present disclosure is configured to move the sanitary module in the vertical direction by the two vertically moving frames, so that the mattress care device can be stored while being hidden in an unused state and can be located while being exposed upward from the mattress during a use time.

The mattress care device of the present disclosure is configured to adjust the height of the sanitary module, so that UV-C irradiation can be performed while the sanitary module is placed as close to the mattress as possible and thus improve the sterilization effect.

The mattress care device of the present disclosure is configured to perform cleaning with the sanitary module moved forward and rearward on the upper surface of the mattress by the moving part, so that cleaning for the entire area of the mattress can be performed, even by a small suction force.

The mattress care device of the present disclosure is configured to provide the through installation part on each edge of the bed frame, so that the vertical ends located at the front end and the rear end of each of the vertically moving frames can be precisely moved upward and downward.

The mattress care device of the present disclosure has the frame elevation part to move the vertically moving frames upward and downward, so that the mattress care device can be automatically operated without manual operation.

The mattress care device of the present disclosure is configured such that the sanitary module is located higher than the upper surface of the mattress during operation of the frame elevation part and the sanitary module is located lower than the upper surface of the mattress during non-operation of the frame elevation part, so that the sanitary module can be prevented from being exposed to the outside during non-use.

The mattress care device of the present disclosure is configured to move the vertically moving frames upward and downward by the ball screw of the frame elevation part, so that the mattress care device can be used in common regardless of the height of the mattress (i.e., used for any mattress height).

The mattress care device of the present disclosure is configured to operate a first ball screw and a second ball screw in conjunction with each other by the interlocking device, so that the first and second ball screws can be operated collectively by the single elevating motor and tilting of the mattress care device occurring due to the difference in speeds by locations of the vertically moving frames can be prevented.

The mattress care device of the present disclosure is configured to store the sanitary module on the storage groove of the storage part, so that exposure of the sanitary module in an unused state can be minimized.

The mattress care device of the present disclosure is configured to move the movable bar of the sanitary module precisely forward and rearward along the two vertically moving frames, so that cleaning can be performed regardless of a longitudinal length of the mattress.

The mattress care device of the present disclosure is configured to move the sterilizing cleaner leftward and rightward along the lower surface of the movable bar, so that cleaning can be performed regardless of a transverse length of the mattress.

The mattress care device of the present disclosure has the sterilizing cleaner, which has the transverse length configured to be shorter than the transverse length of the movable bar and is moved leftward and rightward, so that cleaning for the mattress can be performed by stronger suction force.

The mattress care device of the present disclosure includes the sanitary module including the fan motor and the dust collecting casing to perform cleaning by suctioning and removing debris existing on the surface of the mattress.

The mattress care device of the present disclosure includes the UV-C irradiation part located on the bottom surface of the sanitary module to perform sterilization cleaning for the surface of the mattress.

The mattress care device of the present disclosure includes the moving part including the rack gear and the pinion gear to perform speed control for forward and rearward movement of the sanitary module.

The mattress care device of the present disclosure includes the guide part formed on the facing surfaces between the vertically moving frames, the pinion gear located on each of the opposite ends of the sanitary module can be precisely moved without separation from the rack gear.

The mattress care device of the present disclosure includes the sanitary module of which the opposite ends are respectively placed on the guide parts, so that the sanitary module can be moved without sagging of the opposite ends of the sanitary module.

The mattress care device of the present disclosure includes the storage part having the covering wall, so that an outward exposed portion of the sanitary module can be reduced in a stored state.

Further scope of applicability of the invention will become apparent from the detailed description given hereinafter. However, it should be understood that the detailed description and specific examples, while indicating embodiments of the invention, are given by way of illustration only, since various changes and modifications within the scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention.

FIG. 1 is a perspective view showing a bed with a mattress care device according to an embodiment of the present invention.

FIG. 2 is a sectional view showing an inside structure of the bed with the mattress care device according to an embodiment of the present invention.

FIG. 3 is a perspective view showing the installation of vertically moving frames of the mattress care device according to an embodiment of the present invention while a part of a bed frame is removed.

FIG. 4 is a perspective view showing the bed frame of the mattress care device according to an embodiment of the present invention.

FIG. 5 is a plan view showing the mattress care device according to an embodiment of the present invention.

FIG. 6 is a plan view showing a frame elevation part of the mattress care device according to an embodiment of the present invention.

FIG. 7 is an exploded-perspective view showing the installation of a sanitary module of the mattress care device according to an embodiment of the present invention.

FIG. 8 is an enlarged view showing part “A” of FIG. 7.

FIG. 9 is a plan view showing the installation of the sanitary module of the mattress care device according to an embodiment of the present invention.

FIG. 10 is a main part cut out perspective view showing the installation of a sterilizing cleaner of the sanitary module of the mattress care device according to an embodiment of the present invention.

FIG. 11 is a side sectional view showing an inside structure of the sterilizing cleaner of the sanitary module of the mattress care device according to an embodiment of the present invention.

FIG. 12 is a front sectional view showing the inside structure of the sterilizing cleaner of the sanitary module of the mattress care device according to an embodiment of the present invention.

FIG. 13 is a block diagram schematically showing a control structure performed by a controller of the mattress care device according to an embodiment of the present invention.

FIG. 14 is a stored state of the mattress care device according to an embodiment of the present invention.

FIG. 15 is a perspective view showing the stored state of the mattress care device according to an embodiment of the present disclosure while a part of the bed frame is removed.

FIG. 16 is a side sectional view showing a frame elevation part of the mattress care device according to an embodiment of the present invention.

FIG. 17 is a perspective view showing the vertically moving frames in a raised state for using the mattress care device according to an embodiment of the present invention.

FIG. 18 is a perspective view showing the vertically moving frames in the raised state of the mattress care device according to the embodiment of the present disclosure while a part of the bed frame is removed.

FIG. 19 is a side sectional view showing the vertically moving frames in the raised state for using the mattress care device according to an embodiment of the present invention.

FIG. 20 is a plan view showing a process of cleaning a mattress by the mattress care device according to an embodiment of the present invention.

FIG. 21 is a sectional view showing the inside of the sterilizing cleaner of the mattress care device according to an embodiment of the present invention.

### DETAILED DESCRIPTION OF THE PREFERRED EMBODIMENTS

The present invention will now be described with reference to the accompanying drawings, wherein the same reference numerals have been used to identify the same or similar elements throughout the several views.

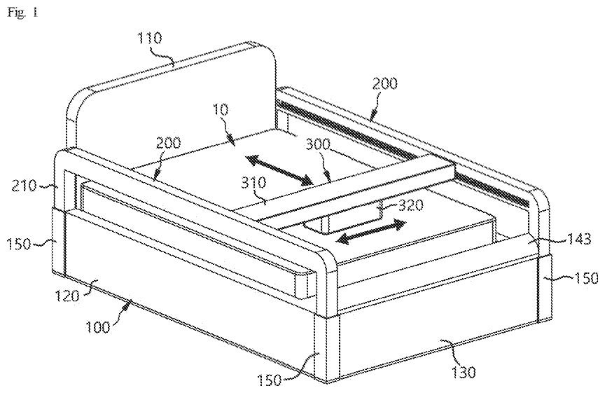

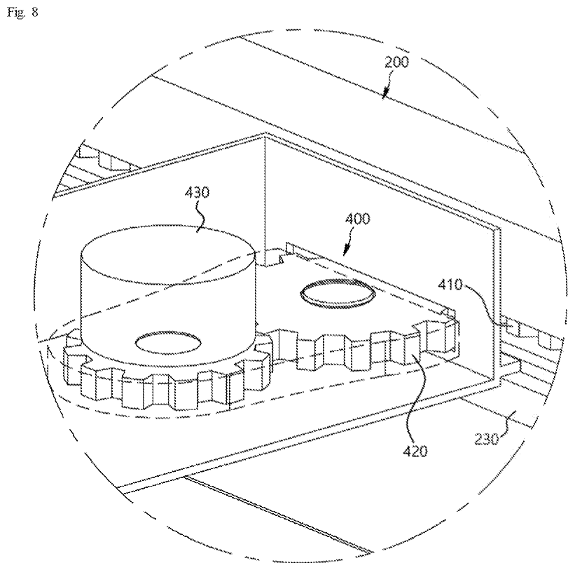

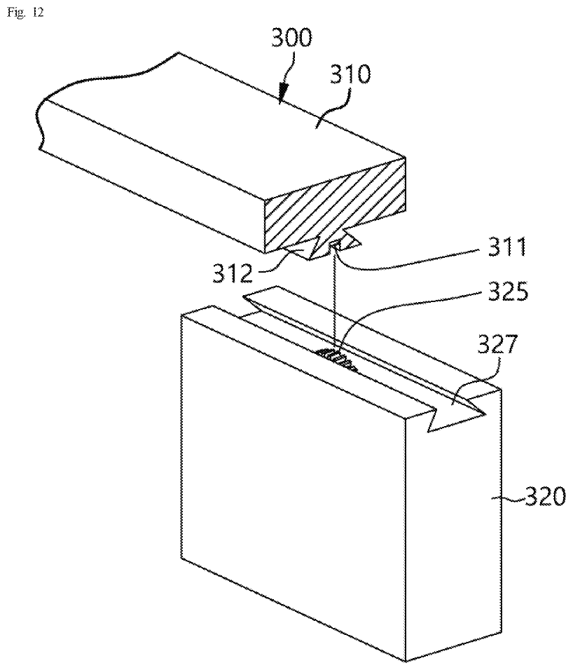

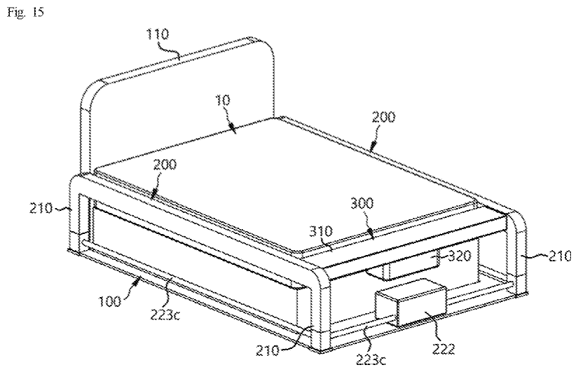

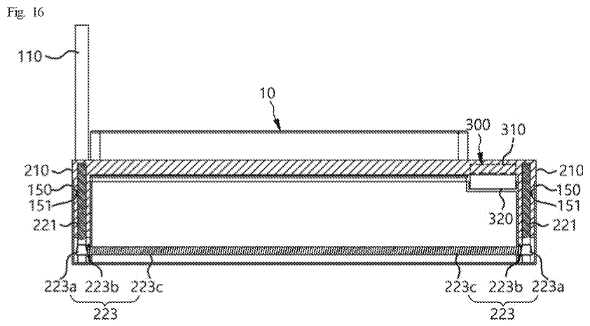

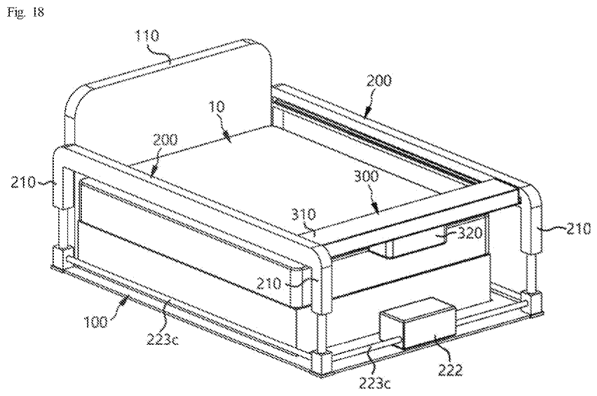

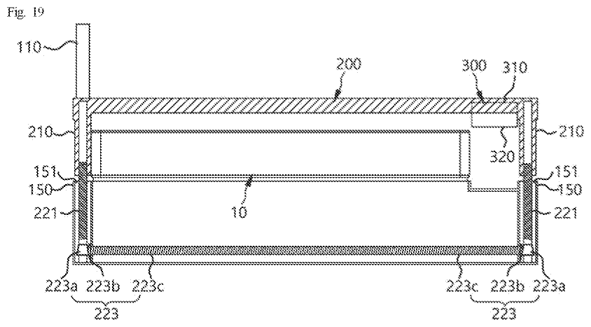

Hereinbelow, a mattress care device according to exemplary embodiments of the present disclosure will be described with reference to FIGS. 1 to 21.

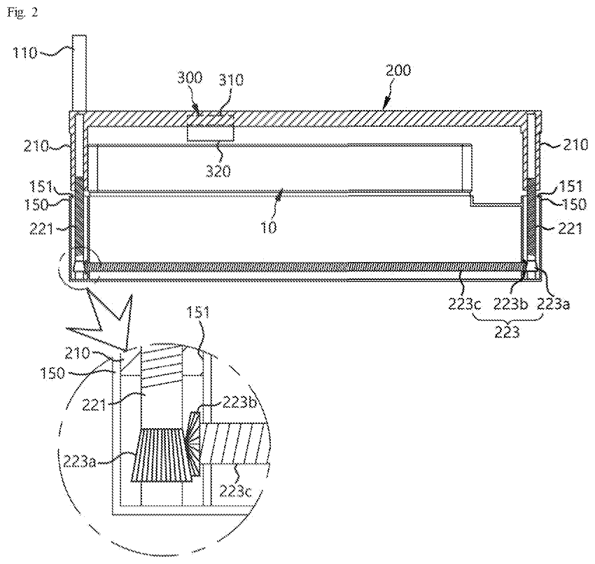

FIG. 1 is a perspective view showing a bed with a mattress care device according to an embodiment of the present disclosure. FIG. 2 is a sectional view showing an inside structure of the bed with the mattress care device according to the embodiment of the present disclosure.

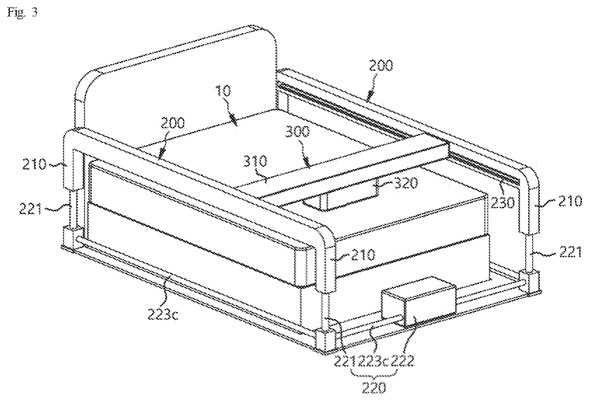

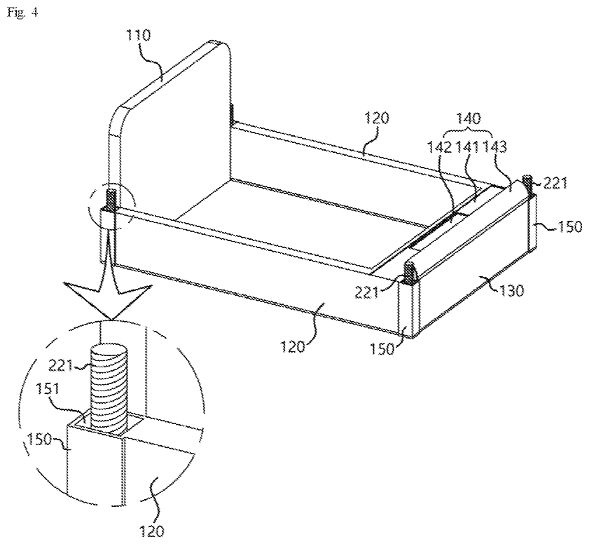

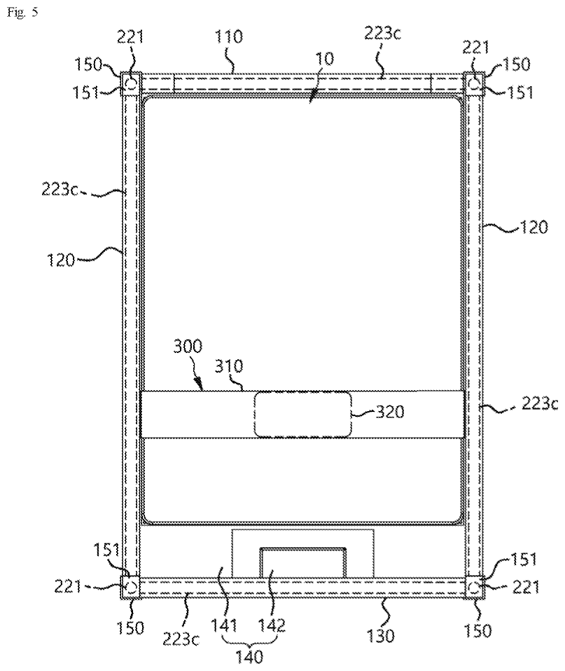

FIG. 3 is a perspective view showing the installation of vertically moving frames of the mattress care device according to the present disclosure while a part of a bed frame is removed. FIG. 4 is a perspective view showing the bed frame of the mattress care device according to the embodiment of the present disclosure. FIG. 5 is a plan view showing the mattress care device according to the embodiment of the present disclosure.

As shown in the drawings, the mattress care device according to the embodiment of the present disclosure may be configured to be located lower than an upper surface of the mattress 10 and to be stored in a hidden state from the user's view when not in use, and the mattress care device may be configured to be located higher than the upper surface of the mattress 10 and to clean the upper surface of the mattress 10 when used.

When a sterilization cleaning is not performed, the mattress care device may be configured to prevent a sterilizing cleaner 320 from being exposed to the outside and to maintain the hidden state of the sterilizing cleaner 320 so as to prevent a user from feeling visual discomfort.

The mattress care device according to the embodiment of the present disclosure will be described as follows.

First, the mattress care device according to the embodiment of the present disclosure may include a bed frame 100.

The bed frame 100 may be provided to form the exterior of the bed, and be configured to accommodate the mattress 10.

The bed frame 100 may be formed in a box shape including a front wall 110 as the head portion of the bed, side walls 120 as the side portions of the bed, and a rear wall 130 as the foot portion of the bed and being open upward.

Furthermore, the bed frame 100 may include a storage part 140 (as shown in FIG. 4).

The mattress 10 may be configured to be accommodated in a space from the head portion to the foot portion in the bed frame 100 (e.g., the front wall 110 to the rear wall 130). The storage part 140 may be provided in a space between the foot portion of the mattress 10 and the rear wall 130 of the bed frame 100 and a sanitary module 300, which is described below, may be stored in the storage part 140.

The storage part 140 may include a storage groove 141 formed by being depressed an upper surface of a rear end of the bed frame 100.

The storage groove 141 may be formed on an upper surface of a front end of the bed frame 100, or may be formed both the upper surfaces of the front and rear ends of the bed frame 100.

Specifically, a cleaner accommodation groove 142 may be formed in the storage groove 141 of the storage part 140 to accommodate the sterilizing cleaner 320 of the sanitary module 300. As the cleaner accommodation groove 142 is additionally provided, the sterilizing cleaner 320 may be stored in a regular location thereof.

In addition, a covering wall 143 may be provided in rear of the storage part 140. When the sanitary module 300 is seated in the storage part 140, the covering wall 143 may cover a rear edge of a movable bar 310 of the sanitary module 300.

The covering wall 143 may be formed in a round shape, but may alternatively have any shape.

Furthermore, a through installation part 150 having a through hole 151 may be provided at each corner of the bed frame 100. The through hole 151 may be formed by penetrating the through installation part 150 in a vertical direction.

The through installation part 150 may be formed of each corner of the bed frame 100. Of course, the through installation part 150 may be provided separately from the bed frame 100 and may be securely coupled to each corner of the bed frame 100.

Next, the mattress care device according to the embodiment of the present disclosure may include two vertically moving frames 200 (e.g., a first vertically moving frame 200 or a and a second vertically moving frame 200). The first and second vertically moving frames 200 may also be designated as first and second moving frames 200. However, any number of vertically moving frames 200 may be used.

The two vertically moving frames 200 may be formed in a bar (i.e., formed of a bar) and may be formed longer than at least a longitudinal length of the mattress 10.

In addition, the two vertically moving frames 200 may be provided to be movable upward and downward on the opposite walls of the bed frame 100, which is transverse to a direction in which the mattress 10 extends.

Specifically, vertical ends 210 of the two vertically moving frames 200 may be provided in a front end and a rear end of each of the two vertically moving frames 200. Each of the vertical ends may pass through the through hole 151 formed in the through installation part 150 of the bed frame 100 in the vertical direction. That is, each of the two vertical moving frames 200 has a center portion that extends substantially in a horizontal direction and two vertical ends (i.e., two ends extending in a vertical direction) extending downward in a substantially vertical direction, transverse to the horizontal direction, towards a frame elevation part 220, which is described below.

The vertical ends 210 may be formed in a shape bent downward from opposite ends of each of the vertically moving frames 200. The vertical ends 210 may be configured to be either coupled to the opposite ends of each vertically moving frame 200 or to form the ends of each vertically moving frame 200.

Therefore, by the vertical ends 210, the two vertically moving frames 200 may be moved upward or downward from the side walls 120 of the bed frame 100.

The vertically moving frames 200 may be configured to be moved upward and downward by a manipulation of a user, or by automatic operation performed by control of a controller 500.

According to the embodiment of the present disclosure, the vertically moving frames 200 may be configured to be moved upward and downward by a frame elevation part 220 (referring to FIG. 3) under the control of the controller 500.

Specifically, the frame elevation part 220 may be configured to raise the vertically moving frames 200 so as to locate the sanitary module 300 higher than the upper surface of the mattress 10 in an operative position, or to lower the vertically moving frames 200 so as to locate the sanitary module 300 lower than the upper surface of the mattress in a non-operative position.

In a cleaning operation, the frame elevation part 220 may be operated to locate the sanitary module 300 higher than the upper surface of the mattress 10, and in a non-cleaning operation, the frame elevation part 220 may be operated to locate the sanitary module 300 lower than the upper surface of the mattress 10.

The frame elevation part 220 may be configured to use a ball screw 221.

Each of the vertical ends 210 may be provided to be moved upward and downward along a ball screw 221. A lower end of the ball screw 221 may be provided to be exposed from a lower portion of the vertical end 210, and the exposed portion of the ball screw 221 may be connected to an elevating motor 222 to be capable of power transmission.

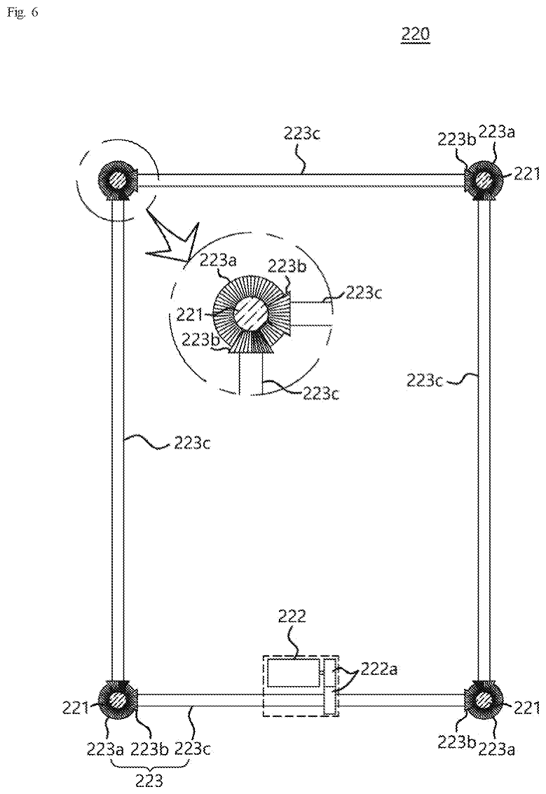

Specifically, the ball screw 221 may be a first ball screw 221 and may connected to a second ball screw 221 by an interlocking device 223 (referring to FIGS. 2 and 6) to be operated in conjunction with each other. When the single elevating motor 222 is driven, both the ball screws 221 may be rotated in the same direction by the interlocking device 223.

The interlocking device 223 may be configured to use a gear, a chain, or a belt.

According to the embodiment of the present disclosure, the interlocking device 223 may be operated while using a power transmission structure of a bevel gear, as an example, or any type of gear.

For example, as shown in FIGS. 2 and 6, the ball screw 221 may include a first bevel gear 223a (e.g., ring gear) and at least one second bevel gear 223b, the first bevel gear 223a engages the at least one second bevel gear 223b such that the first bevel gear 223a and the at least one second bevel gear 223b rotate together. The first bevel gear 223a may engage two separate second bevel gears 223b (e.g., pinion gear), and these two separate second bevel gears 223b may be provided at in directions perpendicular to each other, as shown in FIG. 6.

Two second bevel gears 223b located in facing locations may be connected to each other by a connection shaft 223c. The second bevel gears 223b may be respectively provided at opposite ends of the connection shaft 223c and the second bevel gears 223b may be arranged to be engaged with first bevel gears 223a located in two distinct ball screws 221.

One (driving-side connection shaft) of connection shafts 223c may be configured to receive a driving force while being connected to the elevating motor 222 with a first elevator motor gear 222a (e.g., a bevel gear or a belt) to be capable of power transmission.

When the elevating motor 222 is driven, the driving-side connection shaft 223c may be rotated to rotate the second bevel gears 223b at opposite ends of the connection shaft 223c, whereby the two first bevel gears 223a engaged to the two second bevel gears 223b may be respectively rotated to rotate the two ball screws 221.

In addition, when the two first bevel gears 223a are rotated, two other second bevel gears 223b engaged to the two first bevel gears 223a may be rotated to rotate two other first bevel gears 223a, whereby other two ball screws 221 coupled to the two first bevel gears 223a may be rotated. That is, the mattress care device may include four total first bevel gears 223a and four total connection shafts 223c, with each connection shaft 223c having two second bevel gears 223b at first and second ends thereof, and each first bevel gear 223a may engage two second bevel gears 223b of two different connection shafts 223c, as shown in FIG. 6. Therefore, all of the first bevel gears 223a may be operatively connected together via corresponding second bevel gears 223b, and one of the connection shafts 223c may be rotated by the elevating motor 222 via the first elevating motor gear 222a.

Therefore, the four ball screws 221 may be rotated at the same time, and the vertically moving frames 200 coupled to the four ball screws 221 may be moved upward and downward from the bed frame 100.

Next, the mattress care device according to the embodiment of the present disclosure may include the sanitary module 300.

The sanitary module 300 is a device provided to clean the mattress 10.

The sanitary module 300 may be configured to clean the upper surface of the mattress 10 while being moved along the vertically moving frames 200.

Specifically, when not in use, the sanitary module 300 may be located to be stored in the storage part 140 of the bed frame 100 by the downward movement of the vertically moving frames 200, and in the use time, the sanitary module 300 may be located above the mattress 10 out of the storage part 140 and be moved forward and downward.

The sanitary module 300 may include the movable bar 310 as shown in accompanying FIGS. 5 and 6.

The movable bar 310 may be provided to be movable forward and rearward in a longitudinal direction of the vertically moving frame 200 as opposite ends of the movable bar 310 are respectively connected to the two vertically moving frames 200. The transverse length of the two vertically moving frames 200 may be formed longer than the left to right length of the mattress 10. Further, the dimensions of the two vertically moving frames 200 may be larger than the dimensions of the mattress 10, such that the two vertically moving frames 200 can move the sanitary module 300 over any portion of the mattress 10.

Furthermore, the sanitary module 300 may include the sterilizing cleaner 320.

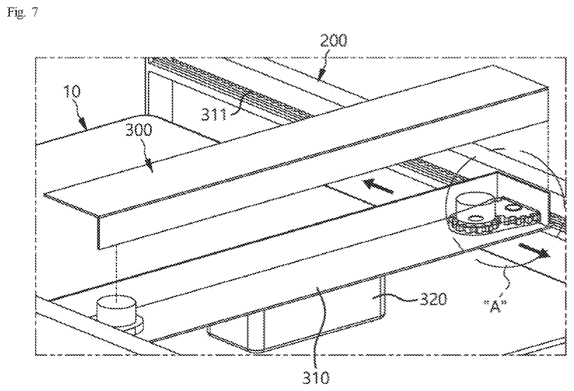

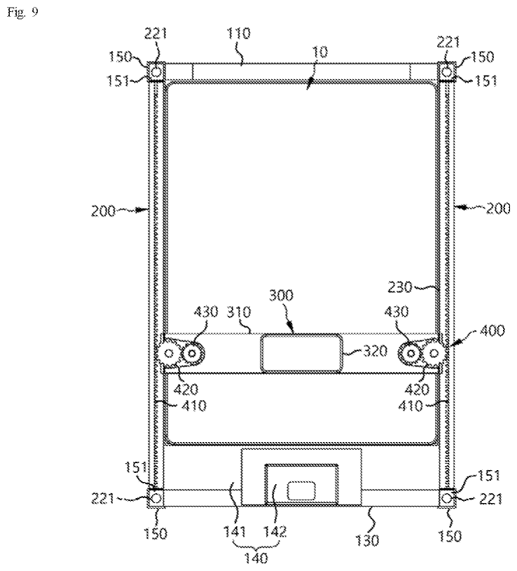

As shown in FIGS. 7 and 9, the sterilizing cleaner 320 may be provided to be movable leftward and rightward along a lower surface of the movable bar 310 and be configured to clean a surface of the mattress 10.

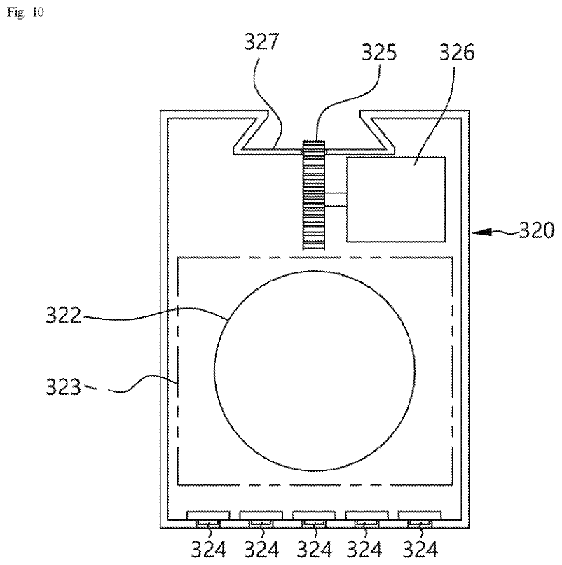

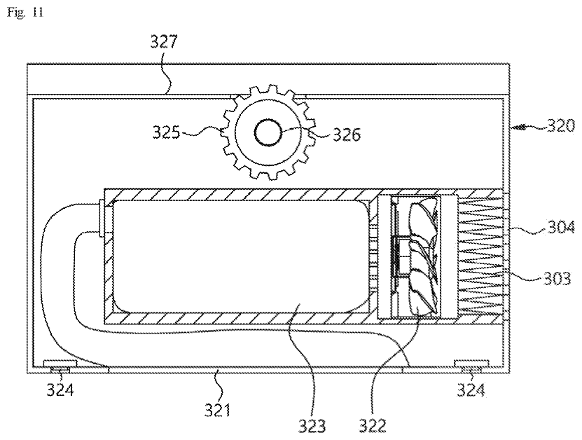

The sterilizing cleaner 320 may be provided to remove debris accumulated on a surface of the mattress 10. That is, the sterilizing cleaner 320 may include at least a vacuum having a fan motor 322 and a dust collecting casing 323, as described below. As shown in FIGS. 10 and 11, an inlet 321 (e.g., intake hole) may be formed on a bottom surface of the sterilizing cleaner 320, and the sterilizing cleaner 320 may include a fan motor 322 and a dust collecting casing 323 therein. Further, the sterilizing cleaner 320 may further include a filter 303, disposed downstream of the fan motor 322, for filtering the air received from the fan motor 322, and an outlet 304 (e.g., discharge hole). The fan motor 322 may provide a vacuum suction force to the inlet 321 and the dust collecting casing 323 may collect debris contained in air suctioned through the inlet 321.

The transverse length of the sterilizing cleaner 320 may be configured to be shorter than the leftward and rightward length of the movable bar 310.

Although not shown in the drawings, the inlet 321 is provided on the bottom surface of the movable bar 310 and the fan motor 322 and the dust collecting casing 323 are provided in the movable bar 310, so that the movable bar 310 may be configured to replace the sterilizing cleaner 320.

However, as the length of the inlet 321 provided in the movable bar 310 is formed longer, the suction force is lowered, so in order to increase the suction force, the size of the fan motor 322 should be increased or the fan motor 322 with more excellent performance should be used. Therefore, considering that manufacturing cost may be increase due to the above-mentioned configuration, it is preferable that an additional sterilizing cleaner 320 is provided.

Furthermore, the sterilizing cleaner 320 may include at least one UV-C irradiation part 324 radiating UV-C. UV-C radiated by the UV-C irradiation part 324 may sterilize the upper surface of the mattress 10.

The UV-C irradiation part 324 may be provided at least one portion in the bottom surface of the sterilizing cleaner 320.

In addition, the UV-C irradiation part 324 may consist of any one of an UV-C lamp and an UV-C LED. According to the embodiment of the present disclosure, the UV-C irradiation part 324 consists of the UV-C LED, as an example.

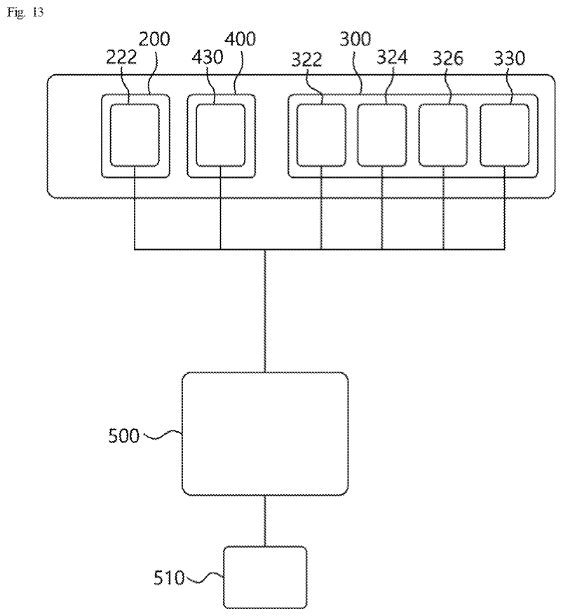

Furthermore, the sanitary module 300 may include a detection part 330 (referring to FIG. 13) provided to determine a direction change location.

The detection part 330 may be configured to detect whether the sanitary module 300 reaches the front wall 110 or the rear wall 130 of the bed frame 100. The detection part 330 may consist of at least one of a sensor and a switch. The controller 500 may control forward and rearward movement of the sanitary module 300 in response to the location information of the sanitary module 300 detected by the detection part 330.

The detection part 330 may be provided in the bed frame 100, (e.g., not in the sanitary module 300).

Meanwhile, the sterilizing cleaner 320 may be configured to be automatically moved along the movable bar 310 by the control of the controller 500.

Therefore, according to the embodiment of the present disclosure, a rack gear 311 may be formed in a moving direction of the sterilizing cleaner 320 on the lower surface of the movable bar 310, and a pinion gear 325 may be provided at the sterilizing cleaner 320 and be operated while being engaged with the rack gear 311. The above-mentioned structure is the same as shown in FIGS. 10 to 12.

A cleaner moving motor 326 may be provided in the sterilizing cleaner 320 to drive the pinion gear 325. The cleaner moving motor 326 may be configured to be controlled by the controller 500.

The sterilizing cleaner 320 may be moved using an actuator or a linear motor, and may be formed with the ball screw structure. That is, the cleaner moving motor 326 may be an actuator or a linear motor formed with a ball screw structure.

The sterilizing cleaner 320 may be configured to be moved on the lower surface of the movable bar 310 while be supported by a guide rail 312.

The guide rail 312 may be formed on any one of facing surfaces between the sterilizing cleaner 320 and the movable bar 310. A guide groove 327 may be formed on another surface of the facing surfaces.

According to the embodiment of the present disclosure, as an example, the guide rail 312 may be formed by protruding from the lower surface of the movable bar 310, and the guide groove 327 may be formed by being depressed on an upper surface of the sterilizing cleaner 320. The guide rail 312 may have an outwardly flared shape (e.g., a triangular shape) with the rack gear 311 being disposed at a center (e.g., cross-sectional center) of the guide rail 312. Further, the guide groove 327 (i.e., female member) may have a shape corresponding to that of the guide rail 312 (i.e., male member), such that the guide rail 312 fits into the guide groove 327, to permit the pinion gear 325 to engage the rack gear 311.

Furthermore, the sanitary module 300 may be configured to be moved along the vertically moving frames 200 while being supported by being supported by guide parts 230 (FIG. 3).

The guide parts 230 may be provided to inwardly protrude from the facing surfaces between the two vertically moving frames 200. The opposite ends of the movable bar 310 of the sanitary module 300 may be configured to be respectively placed on the guide parts 230.

Next, the mattress care device according to the embodiment of the present disclosure may include a moving part 400.

The moving part 400 may be configured to move the sanitary module 300 forward and rearward.

The moving part 400 may include a rack gear 410 and a pinion gear 420, as shown in FIG. 9.

The rack gear 410 may be formed on each of the facing surfaces between the two vertically moving frames 200 in a longitudinal direction of the vertically moving frame 200. The pinion gear 420 may be provided at each of the opposite ends of the movable bar 310 of the sanitary module 300 to be engaged with the rack gear 410.

The pinion gear 420 may be rotated by receiving a driving force of a module moving motor 430. The pinion gear 420 may be moved along the rack gear 410.

Specifically, the module moving motor 430 may be located in the movable bar 310, and may be directly connected to the pinion gear 420 or be provided to be capable of power transmission by a belt or a chain.

Next, the mattress care device according to the embodiment of the present disclosure may include the controller 500.

As shown in FIG. 13, the controller 500 may be configured to control operation of the vertically moving frames 200 and operation of the sanitary module 300.

The controller 500 may be configured to control each of the elevating motor 222 for operating the vertically moving frames 200, the module moving motor 430 for operating the sanitary module 300, and the cleaner moving motor 326 for operating the sterilizing cleaner 320.

The controller 500 may include an operation part 510 for manual operation. The operation part 510 may be provided at the bed frame 100 or at the sanitary module 300.

Preferably, the operation part 510 may be provided at one portion on an exterior surface of the bed frame 100, and the controller 500 may be provided at the bed frame 100 or the sanitary module 300.

The controller 500 may be controlled by a mobile terminal of a user, home network system, or other online controlling.

Specifically, the controller 500 may be controlled such that a movement speed of the sterilizing cleaner 320 is changed in response to operation of the UV-C irradiation part 324.

When the UV-C irradiation part 324 is operated to irradiate UV-C, the sterilizing cleaner 320 or the movable bar 310 is controlled to be moved at a moving speed lower than a moving speed of the UV-C irradiation part 324 in non-radiating state. Therefore, UV-C is sufficiently radiated on the mattress 10 to provide the sterilization effect. When only the suction cleaning is performed without the sterilization cleaning, cleaning time may be reduced.

Furthermore, when the controller 500 receives a sensing signal (or, switch signal) from the detection part 330, the controller 500 may control to stop the forward and rearward movement of the sanitary module 300 or to move the sanitary module 300 rearward.

Hereinbelow, a cleaning process of the mattress 10 performed using the above-mentioned mattress care device of the present disclosure will be described in detail.

First, when cleaning for the mattress 10 is not performed, the vertically moving frames 200 are maintained while being placed on upper surfaces of the side walls 120 of the bed frame 100.

The movable bar 310 of the sanitary module 300 may be maintained while being placed on the storage groove 141 formed in the storage part 140 of the bed frame 100. The sterilizing cleaner 320 of the sanitary module 300 may be stored while being accommodated in the cleaner accommodation groove 142 formed in the storage part 140.

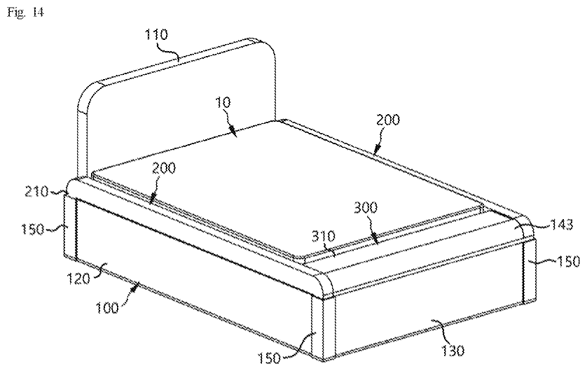

The upper surface of the sterilizing cleaner 320 may be stored in the cleaner accommodation groove 142 while being hidden by the movable bar 310. Therefore, the sterilizing cleaner 320 may be prevented from being exposed outward, thus visual discomfort of the user may be prevented. The above-mentioned structure is the same as shown in FIGS. 14 to 16.

When a preset cleaning cycle in an initial state arrives or a manipulation of the user via the operation part 510 is generated and a cleaning command is performed, the controller 500 may control operation of the frame elevation part 220 and upward movement of the vertically moving frames 200.

As the elevating motor 222 of the frame elevation part 220 is driven, the ball screw 221 located at each corner of the bed frame 100 is rotated, whereby the vertical ends 210 coupled to the ball screw 221 may be moved upward to move the two vertically moving frames 200 upward.

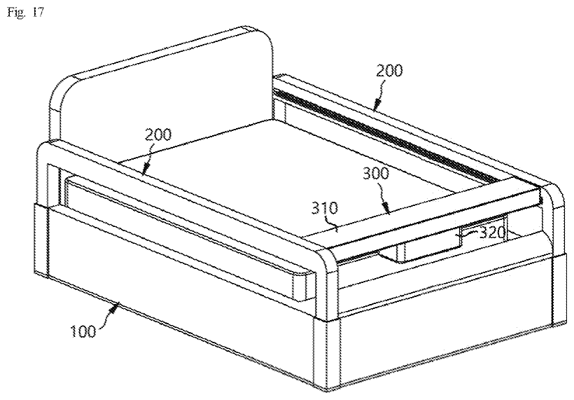

In addition, when the two vertically moving frames 200 are moved upward, the sanitary module 300 may also be moved upward together with the two vertically moving frames 200 and be separated from the storage part 140. The above-mentioned structure is the same as shown in FIGS. 17 to 19.

The upward movement of the two vertically moving frames 200 is performed until the bottom of the sterilizing cleaner 320 is located higher than the upper surface of the mattress 10. The distance of the upward movement of the vertically moving frames 200 may be preset by the user in advance or be checked in real time using a sensor (e.g., encoder, etc.).

When the two vertically moving frames 200 are fully raised by the above process, as the cleaner moving motor 326 and the module moving motor 430 are driven by control of the controller 500, the movable bar 310 and the sterilizing cleaner 320 of the sanitary module 300 may be moved.

The controller 500 may controls the movable bar 310 of the sanitary module 300 to be located at the rear (i.e., foot side) or the front (i.e., head side) of the bed frame 100, and control the sterilizing cleaner 320 to be located at one side of the movable bar 310. The location of the movable bar 310 and the sterilizing cleaner 320 may be an initial location (i.e., preset location) that is preset to automatically perform cleaning.

When the sanitary module 300 is returned to the preset location, the controller 500 may control the module moving motor 430 of the moving part 400 to be operated and the movable bar 310 to be moved forward.

Furthermore, in the forward movement of the movable bar 310, the controller 500 may control the fan motor 322 of the sterilizing cleaner 320 to be operated together.

Therefore, debris existing on the upper surface of the mattress 10 may be suctioned into the sterilizing cleaner 320 and collected in the dust collecting casing 323.

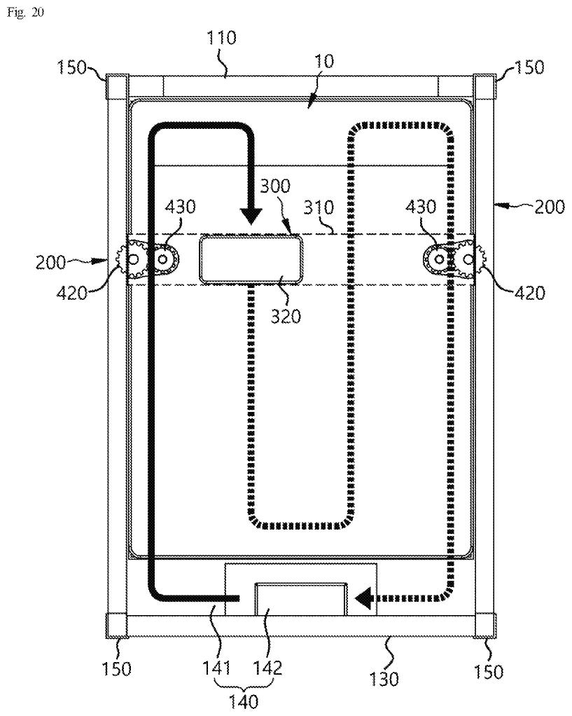

In addition, the controller 500 may control the sterilizing cleaner 320 to be moved in a longitudinal direction of the movable bar 310 by operating the cleaner moving motor 326 of the sterilizing cleaner 320 in the forward movement of the movable bar 310, as shown in FIG. 20.

Therefore, the sterilizing cleaner 320 may suction debris, via the fan motor 322 and the dust collection casing 323, on the upper surface of the mattress 10 while being supported by the guide rail 312 and moved from one side of the movable bar 310 to another side thereof.

The sterilizing cleaner 320 may be operated to be moved when the movable bar 310 completely performs forward movement or rearward movement. The movement distance of the sterilizing cleaner 320 may be moved by a distance considering the suction force of the sterilizing cleaner 320. The movement of the movable bar 310 and the movement of the sterilizing cleaner 320 may be performed in order, thus cleaning for the entire area of the mattress 10 may be uniformly performed.

Both the transverse movement of the sterilizing cleaner 320 and the longitudinal movement of the movable bar 310 may be controlled to be operated at the same time. Then, considering moving paths of the sterilizing cleaner 320 and the movable bar 310, the controller may control each speed of the sterilizing cleaner 320 and the movable bar 310 or be control forward and rearward movements of the movable bar 310 to be repeated.

The controller 500 controls a moving direction of the movable bar 310 to be changed on the basis of the sensing information of the detection part 330.

Meanwhile, during the cleaning for the mattress 10 as described above, the UV-C irradiation part 324 of the sterilizing cleaner 320 may be operated by a presetting or manipulation of the user.

Of course, the controller may control the UV-C irradiation part 324 to be separately operated without double operation with the fan motor 322, and to be operated only in the forward movement or the rearward movement of the sterilizing cleaner 320.

As described above, when the UV-C irradiation part 324 is operated, UV-C may be radiated to the mattress 10 and harmful insects, viruses and/or bacteria in the mattress 10 may be removed or destroyed (i.e., eradicated).

Specifically, when the UV-C irradiation part 324 of the sterilizing cleaner 320 is operated, the controller may control the sterilizing cleaner 320 to be moved slower than when only the fan motor 322 is operated. In the operation of the UV-C irradiation part 324, as the sterilizing cleaner 320 is controlled to be moved slower, the sterilization effect using UV-C may be sufficiently secured (i.e., the sterilization effect may be improved or be more effective).

Meanwhile, when the cleaning of the mattress 10 as described above is complete, the sanitary module 300 may be returned to the location where the storage part 140 is located.

When the sanitary module 300 is returned to the preset location, the elevating motor 222 of the frame elevation part 220 may be driven to reverse-rotate the ball screw 221 located at each corner of the bed frame 100.

Therefore, the vertical end 210 coupled to the ball screw 221 may be moved downward, the two vertically moving frames 200 may be moved downward, the movable bar 310 of the sanitary module 300 may be placed within the storage groove 141 of the storage part 140, and the sterilizing cleaner 320 of the sanitary module 300 may be accommodated in the cleaner accommodation groove 142.

Therefore, the sterilizing cleaner 320 stored in the storage part 140 may not be exposed to the outside of the bed and be stored in a state of being hidden from view.

The mattress care device of the present disclosure may be configured to automatically perform the sterilization cleaning and the vacuum cleaning of the mattress 10, whereby continuous management of the mattress 10 may be performed.

Furthermore, the mattress care device of the present disclosure may include the sterilizing cleaner 320 of the sanitary module 300 stored in the storage part 140 without exposure to the outside when the cleaning of the mattress 10 is not performed, so that the user cannot feel discomfort.

Furthermore, the mattress care device of the present disclosure may include the vertically moving frames 200 located at the opposite sides of the mattress 10 and a portion of the vertically moving frames 200 having the rack gear 410 of the moving part 400 is stored without exposure to the outside, so that contamination or discomfort caused by exposure of the portion may be prevented.

Furthermore, the mattress care device of the present disclosure may be configured to form the moving structure of the vertically moving frames 200 using the ball screws 221, so that the height of the vertically moving frames 200 may be adjusted and thus the mattress care device may be used in common regardless of the thickness of the mattress 10.

Meanwhile, it may not be limited that the mattress care device of the present disclosure is embodied only into the above-described structure.

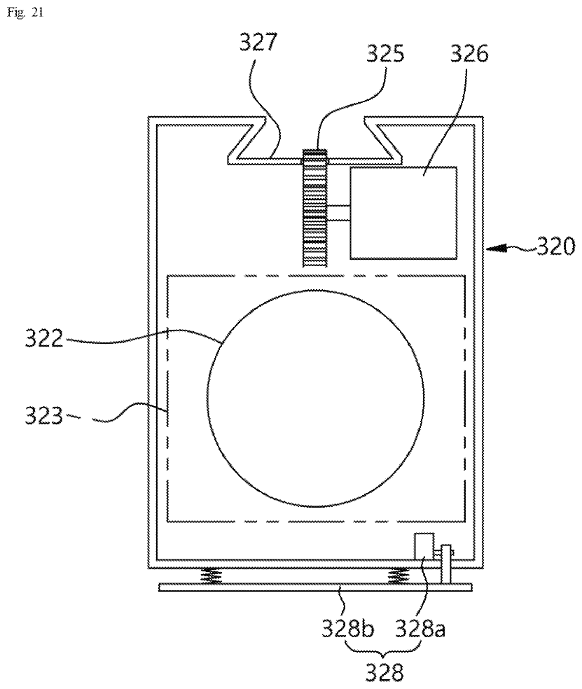

For example, the mattress care device of the present disclosure may include a vibration part 328 in the sterilizing cleaner 320, as shown in FIG. 21. As the vibration part 328 is additionally provided, a surface of the mattress 10 where the sterilizing cleaner 320 passes may be hit, and debris on the surface of the mattress 10, such as dust, may be easily fallen and suctioned.

The vibration part 328 may include a vibration source 328a, such as an ultrasonic vibrator or a vibration motor, and a vibration plate 328b vibrated vertically by operation of the vibration source 328a, as shown in FIG. 21.

The two vertically moving frames 200 of the mattress care device of the present disclosure may not be located at the opposite side walls of the bed frame 100, and may be located at locations where the front wall 110 and the rear wall 130 of the bed frame 100 are located.

Then, the movable bar 310 of the sanitary module 300 may be arranged in the longitudinal direction (front to rear direction) of the mattress 10 and may be configured to be moved in the transverse direction (left to right direction) of the mattress 10, and the storage part 140 may be provided on any one of the opposite side walls 120 of the bed frame 100.

As described above, the mattress care device of the present disclosure may be embodied in various shapes.

The present invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A mattress care device, comprising:
a bed frame configured to accommodate a mattress;
a first vertically moving frame movably attached to a first side of the bed frame;
a second vertically moving frame movably attached to a second side of the bed frame opposite to the first side of the bed frame, the first vertically moving frame and the second vertically moving frame being configured to be vertically movable from the bed frame;
a sanitary module having a first end connected to the first vertically moving frame and a second end connected to the second vertically moving frame, the sanitary module being configured to move along the first vertically moving frame and the second vertically moving frame; and
a moving part configured to move the sanitary module along the first vertically moving frame and the second vertically moving frame.

2. The mattress care device of claim 1, wherein the bed frame includes four corners, and
wherein each of the four corners of the bed frame includes a through installation part having a vertical through hole.

3. The mattress care device of claim 2, wherein each of the first vertically moving frame and the second vertically moving frame includes two vertical ends, and
wherein each vertical end passes through the vertical through hole of a respective one of the through installation parts.

4. The mattress care device of claim 1, further comprising a frame elevation part configured to raise and lower the first vertically moving frame and the second vertically moving frame.

5. The mattress care device of claim 4, wherein the frame elevation part is configured to:
move the first vertically moving frame and the second vertically moving frame upwardly, so that the sanitary module is located higher than an upper surface of the mattress, in response to a cleaning operation being performed, and
move the first vertically moving frame and the second vertically moving frame downwardly, so that the sanitary module is located lower than an upper surface of the mattress, in response to the cleaning operation not being performed.

6. The mattress care device of claim 5, wherein the bed frame includes a storage part configured to selectively store the sanitary module, in response to the cleaning operation not being performed.

7. The mattress care device of claim 4, wherein the frame elevation part includes:
a plurality of ball screws, each ball screw being arranged in a center portion of a respective vertical end in a longitudinal direction of the vertical end, each ball screw including a first bevel gear at a lower end of the ball screw; and
an elevating motor connected to the first bevel gears, the elevating motor being configured to rotate the first bevel gears, to rotate the plurality of ball screws and raise or lower the first vertically moving frame and the second vertically moving frame.

8. The mattress care device of claim 7, wherein each ball screw is connected to the elevation motor via at least one connection shaft and at least one second bevel gear.

9. The mattress care device of claim 1, wherein the sanitary module includes a movable bar having a first end and a second end,
wherein the first end of the movable bar is connected to the first vertically moving frame,
wherein the second end of the movable bar is connected to the second vertically moving frame, and
wherein the movable bar is configured to be movable along a longitudinal direction of the first vertically moving frame and the second vertically moving frame.

10. The mattress care device of claim 9, wherein the sanitary module further includes a sterilizing cleaner configured to be movable, in a horizontal direction perpendicular to the longitudinal direction, on a lower surface of the movable bar.

11. The mattress care device of claim 10, wherein the movable bar includes a rack gear formed on the lower surface of the movable bar, and
wherein the sterilizing cleaner includes:
a pinion gear engaging the rack gear of the movable bar; and
a cleaner moving motor engaged with the pinion gear of the sterilizing cleaner, the cleaner moving motor being configured to drive the pinion gear of the sterilizing cleaner to move the sterilizing cleaner with respect to the movable bar.

12. The mattress care device of claim 11, wherein the sterilizing cleaner further includes a guide groove, and
wherein the movable bar further includes a guide rail engaged with the guide groove of the sterilizing cleaner, the guide rail including the rack gear at a center of the guide rail.

13. The mattress care device of claim 11, wherein the sterilizing cleaner includes an ultraviolet-c (UV-C) irradiation part on a bottom surface of the sterilizing cleaner, the UV-C irradiation part being configured to irradiate UV-C to the mattress.

14. The mattress care device of claim 10, wherein the sterilizing cleaner includes:
an inlet formed on a bottom surface of the sterilizing cleaner;
a fan motor; and
a dust collecting casing,
wherein the fan motor is configured to generate a vacuum suction force to the inlet of the sterilizing cleaner, and
wherein the dust collecting casing is configured to collect debris contained in air suctioned through the inlet of the sterilizing cleaner.

15. The mattress care device of claim 1, wherein the moving part includes:
a plurality of rack gears, each rack gear being provided on an interior surface of a respective one of the first vertically moving frame and the second vertically moving frame, and each rack gear extending in a longitudinal direction of the respective one of the first vertically moving frame and the second vertically moving frame;
a pinion gear provided at each of the first end and the second end of the sanitary module and engaged with a respective one of the plurality of rack gears; and
a module moving motor configured to drive the pinion gears to move the sanitary module along the plurality of rack gears.

16. A mattress care device, comprising:
a bed frame configured to accommodate a mattress;
a first vertically moving frame movably attached to a first side of the bed frame;
a second vertically moving frame movably attached to a second side of the bed frame opposite to the first side of the bed frame;
a frame elevation part including:
a plurality of ball screws, each ball screw being arranged in a respective one of the first vertically moving frame and the second vertically moving frame, each ball screw including a first bevel gear at a lower end of the ball screw; and
an elevating motor connected to the first bevel gears, the elevating motor being configured to rotate the first bevel gears, to rotate the plurality of ball screws and raise or lower the first vertically moving frame and the second vertically moving frame; and
a sanitary module having a first end connected to the first moving vertically frame and a second end connected to the second vertically moving frame and configured to:
be movable along the first and second vertically moving frames; and
irradiate ultraviolet-c (UV-C) light to the mattress and/or vacuum the mattress.

17. The mattress care device of claim 16, wherein each ball screw is connected to the elevation motor via at least one connection shaft and at least one second bevel gear.

18. The mattress care device of claim 16, wherein the sanitary module includes a movable bar having a first end and a second end,
wherein the first end of the movable bar is connected to the first vertically moving frame,
wherein the second end of the movable bar is connected to the second vertically moving frame, and
wherein the movable bar is configured to be movable along a longitudinal direction of the first vertically moving frame and the second vertically moving frame.

19. The mattress care device of claim 18, wherein the sanitary module further includes a sterilizing cleaner,
wherein the movable bar includes a rack gear formed on a lower surface of the movable bar, and
wherein the sterilizing cleaner includes:
a pinion gear engaging the rack gear of the movable bar; and
a cleaner moving motor engaged with the pinion gear of the sterilizing cleaner, the cleaner moving motor being configured to drive the pinion gear of the sterilizing cleaner to move the sterilizing cleaner with respect to the movable bar.

20. The mattress care device of claim 16, further comprising a moving part configured to move the sanitary module along the first and second vertically moving frames, wherein the moving part includes:
a plurality of rack gears, each rack gear being provided on an interior surface of a respective one of the first vertically moving frame and the second vertically moving frame, and each rack gear extending in a longitudinal direction of the respective one of the first vertically moving frame and the second vertically moving frame;
a pinion gear provided at each of the first end and the second end of the sanitary module and engaged with a respective one of the plurality of rack gears; and
a module moving motor configured to drive the pinion gears to move the sanitary module along the plurality of rack gears.
